(19) United States
(12) Patent Application Publication
(10) Pub. No.: US 2007/0124827 A1
(43) Pub. Date: May 31, 2007

(54) Type 2 diabetic non-human mammals and methods of use

(75) Inventors: Ji-Won Yoon, Rockville, MD (US); Chungja Yoon, Rockville, MD (US)

Correspondence Address: MCDERMOTT WILL & EMERY LLP, 28 STATE STREET, BOSTON, MA, 02109-1775, US

(73) Assignee: Biotech Institute for International Innovation, Inc., Kyonggi-do (KR)

(21) Appl. No.: 11/545,167

(22) Filed: Oct. 10, 2006

Related U.S. Application Data
(60) Provisional application No. 60/725,917, filed on Oct. 11, 2005.

(51) Int. Cl. A01K 67/027

(52) U.S. Cl. 800/18

(57) ABSTRACT

The invention provides a T2D mouse comprising a progeny from mating a C57BL/6 mouse and a DBA/2 mouse and exhibiting a blood glucose level of at least about 200 mg/dl, wherein the mouse is a non-human mammalian model predictive of human type 2 diabetes. The T2D mouse also can exhibit impairment of glucose tolerance, normal or increased insulin production, impaired adipocyte or muscle glucose transport or decreased expression of at least one polypeptide involved in insulin action. Further provided is a method of producing a non-human mammal predictive of type 2 diabetes. The method includes mating a C57BL/6 mouse with a DBA/2 mouse, and backing crossing offspring of the mating with a parental DBA/2 mouse to produce a progeny exhibiting a blood glucose level of at least about 200 mg/dl that is predictive of human type 2 diabetes. A method of screening for a therapeutic agent for use in treating type 2 diabetes also is provided. The method includes: (a) administering a compound to the mouse of claim 1, and (b) screening the mouse for a reduced symptom of type 2 diabetes, thereby identifying a therapeutic agent for use in treating type 2 diabetes.

### BACKGROUND OF THE INVENTION

This invention relates generally to the treatment of diabetes and, more specifically to non-human mammalian models of diabetes.

In an individual with normal regulation of blood glucose, the pancreatic hormone insulin is secreted in response to increased blood sugar levels. Increased blood glucose generally occurs following a meal and results in insulin action on peripheral tissues such as skeletal muscle and fat. Insulin stimulates cells of these peripheral tissues to actively take up glucose from the blood and convert it to forms for storage. This process is also referred to as glucose disposal. The levels of blood glucose vary from low to normal to high throughout the day within an individual, depending upon whether the person is in the fasting, intermediate, or fed state. These levels are also referred to as hypoglycemia, euglycemia and hyperglycemia, respectively. In the diabetic individual, these changes in glucose homeostasis are disregulated due to either faulty insulin secretion or action, resulting in a chronic state of hyperglycemia.

Diabetes mellitus is a common disorder, with a prevalence of about 4-5%. The risk of developing diabetes increases with increased weight, with as many as 90% of adult onset diabetic patients being obese. Therefore, due to the high incidence of obese adults, the incidence of adult onset diabetes is increasing worldwide. Diabetes mellitus is classified into three major forms. Type 2 diabetes is one form and is also referred to as non-insulin dependent diabetes (NIDDM) or adult-onset diabetes. Type 1 diabetes is the second form and is referred to as insulin-dependent diabetes (IDDM). The third type of diabetes is genetic and is due to mutations in genes controlling pancreatic islet beta (β) cell function. Although the diagnosis of diabetes is based on glucose measurements, accurate classification of all patients is not always possible. Type 2 diabetes is more common among adults and type 1 diabetes dominates among children and teenagers.

Diabetes mellitus of both types 1 and 2 are associated with a shortened life expectancy as well as other complications such as vascular disease and atherosclerosis. Long-term management of diabetes to prevent late complications often includes insulin therapy regardless of whether the patients are classified as type 1 or type 2.

Diabetes mellitus type 2 is a metabolic disorder. The onset of the disease is most common in middle age and later life. Type 2 is often associated with obesity and hypertension, and with the conditions insulin resistance and PCOS or Syndrome X. It is also associated with haemochromatosis, acromegaly, Cushing's syndrome and a number of other endocrinological disorders. Methods of treating type 2 diabetes consist of controlling the symptoms by diet and exercise and, for those with impaired insulin production, insulin maintenance therapy.

There are several animal models for the study of diabetes. The C57BL/6KsJ ob/ob or db/db mouse model is considered as a type 2 diabetes model. These mouse models have ob (obese) gene mutation (ob/ob mouse) or leptin receptor gene mutation (db/db mouse) and develop diabetes spontaneously between 1 to 2 months of age. The phenotypes for these models are hyperglycaemia, obesity and insulin resistance with hyperinsulinemia and impaired pancreatic β-cell functions at late stage, which are the characteristics of human type 2 diabetes. However, this animal develops diabetes at early age and shows obesity, which are not often common in human diabetes.

Zuker rat, GK rat and Otsuka Long-Evans Tokushima rat are considered diabetic rat models. However, these rat models show inconsistent phenotype of diabetes. Therefore, these models lack reliability for use as credible models for human type 2 diabetes.

Thus, there exists a need for an accurate and reliable non-human mammalian model of type 2 diabetes that more closely resembles the human condition. The present invention satisfies this need and provides related advantages as well.

### SUMMARY OF THE INVENTION

The invention provides a T2D mouse comprising a progeny from mating a C57BL/6 mouse and a DBA/2 mouse and exhibiting a blood glucose level of at least about 200 mg/dl, wherein the mouse is a non-human mammalian model predictive of human type 2 diabetes. The T2D mouse also can exhibit impairment of glucose tolerance, normal or increased insulin production, impaired adipocyte or muscle glucose transport or decreased expression of at least one polypeptide involved in insulin action. Further provided is a method of producing a non-human mammal predictive of type 2 diabetes. The method includes mating a C57BL/6 mouse with a DBA/2 mouse, and backing crossing offspring of the mating with a parental DBA/2 mouse to produce a progeny exhibiting a blood glucose level of at least about 200 mg/dl that is predictive of human type 2 diabetes. A method of screening for a therapeutic agent for use in treating type 2 diabetes also is provided. The method includes: (a) administering a compound to the mouse of claim 1, and (b) screening the mouse for a reduced symptom of type 2 diabetes, thereby identifying a therapeutic agent for use in treating type 2 diabetes.

### BRIEF DESCRIPTION OF THE DRAWINGS

FIG. 1 shows the incidence of diabetes in T2D mice.

FIG. 2 shows body weight and blood glucose level in T2D mice.

FIG. 3 shows glucose tolerance and insulin resistance in T2D mice at 10 wks of age.

FIG. 4 shows glucose tolerance and insulin resistance in T2D mice at 30 wks of age.

FIG. 5 shows insulin immunohistostaining of pancreatic islet from T2D mice at 30 wks of age.

FIG. 6 shows plasma C-peptide level in T2D mice at 30 wks of age.

FIG. 7 shows glucose transport in adipocytes from T2D mice at 10 wks of age.

FIG. 8 shows glucose transport in adipocytes from T2D mice at 30 wks of age.

FIG. 9 shows glucose transport in soleus muscle from T2D mice at 10 wks of age.

FIG. 10 shows glucose transport in soleus muscle from T2D mice at 30 wks of age.

FIG. 11 shows age-dependent expression of proteins required for insulin action in adipose tissue from T2D mice.

FIG. 12 shows age-dependent expression of proteins required for insulin action in soleus muscle from T2D mice.

### DETAILED DESCRIPTION OF THE INVENTION

The invention is directed to a non-human mammalian model that reliably mimics human type 2 diabetes. The non-human mammal exhibiting human type 2 diabetes exhibits glucose levels of about 250 mg/dl. The non-human mammalian model also exhibits other characteristics of type 2 diabetes such as impairment of glucose tolerance, insulin resistance and impaired glucose transport. The mammalian model of the invention is useful for optimizing therapeutic treatments and for identifying therapeutic compounds that can treat human type 2 diabetes.

As used herein, the term “diabetes” is intended to mean the diabetic condition known as diabetes mellitus. Diabetes mellitus is a chronic disease characterized by relative or absolute deficiency of insulin which results in glucose intolerance. The term is intended to include all types of diabetes mellitus, including, for example, type I, type II, and genetic diabetes. Type I diabetes is also referred to as insulin dependent diabetes mellitus (IDDM) and also includes, for example, juvenile-onset diabetes mellitus. Type I diabetes is primarily due to the destruction of pancreatic β-cells. Type II diabetes mellitus is also known as non-insulin dependent diabetes mellitus (NIDDM) and is characterized, in part, by impaired insulin releast following a meal. Insulin resistance can also be a factor leading to the occurrence of type II diabetes mellitus. Genetic diabetes is due to mutations which interfere with the function and regulation of β-cells.

Diabetes is characterized as a fasting level of blood glucose greater than or equal to about 140 mg/dl or as a plasma glucose level greater than or equal to about 200 mg/dl as assessed at about 2 hours following the oral administration of a glucose load of about 75 g. The term “diabetes” is also intended to include those individuals with hyperglycemia, including chronic hyperglycemia and impaired glucose tolerance. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes mellitus.

The term treating refers to an amelioration of a clinical symptom indicative of diabetes. Amelioration of a clinical symptom includes, for example, a decrease in blood glucose levels or an increase in the rate of glucose clearance from the blood in the treated individual compared to pretreatment levels or to an individual with diabetes. The term “treating” also includes an induction of a euglycemic response in the individual suffering from disregulated hyperglycemia. Euglycemia refers to the range of blood glucose levels clinically established as normal, or as above the range of hypoglycemia but below the range of hyperglycemia. Therefore, a euglycemic response refers to the stimulation of glucose uptake to reduce the plasma glucose concentration to normal levels. For most adults, this level corresponds to the range in concentration of about 60-105 mg/dL of blood glucose and preferably between about 70-100 mg/dL, but can vary between individuals depending on, for example, the sex, age, weight, diet and overall health of the individual. Effective treatment of a diabetic individual, for example, would be a reduction in that individual's hyperglycemia, or elevated blood glucose levels, to normalized or euglycemic levels, with this reduction directly resulting from secretion of insulin. Alternatively, effective treatment would be a reduction in fasting blood glucose to levels less than or equal to about 140 mg/dL.

The term treating also is intended to include the reduction in severity of a pathological condition or a chronic complication which is associated with diabetes. Such pathological conditions or chronic complications are listed in Table 1 and include, for example, muscle wasting, ketoacidosis, glycosuria, polyuria, polydipsia, diabetic microangiopathy or small vessel disease, atherosclerotic vascular disease or large vessel disease, neuropathy and cataracts.

TABLE 1
Pathological Conditions Associated with Diabetes

Kidney
Glomerular microangiopathy
Diffuse glomerulosclerosis
Nodular glomerulosclerosis (Kimmel-stiel-Wilson disease)
Urinary infections
Acute pyelonephritis
Renal Failure
Necrotizing papillitis
Emphysematous pyelonephritis
Glycogen nephrosis (Armanni-Ebstein lesion)
Eye
Retinopathy
Nonproliferative retinopathy: capillary Microaneurysms,
retinal edema exudates, and hemorrhages
Proliferative retinopathy: proliferation of small vessels
Visual Failure
hemorrhage fibrosis, retinal detachment
Cataracts
Transient refractive errors due to osmotic changes in lens
Glaucoma due to proliferation of vessels in the iris
Infections
Nervous System
Cerebrovascular atherosclerotic disease: strokes, death
Peripheral neuropathy; peripheral sensory and motor cranial, autonomic
Skin
Infections: folliculitis leading to carbuncles
Necrobiosis lipoidica diabeticorum: due to microangiopathy
Xanthomas: secondary to hyperlipidemia
Cardiovascular system
Coronary atherosclerosis: myocardial infarction, death
Peripheral atherosclerosis: limb ischemia, gangrene
Reproductive system
Increased fetal death rate (placental disease, neonatal respiratory
distress syndrome, infection)
General
Increased susceptibility to infection
Delayed wound healing

Additional complications also include, for example, a general increased susceptibility to infection and wound healing. The term “treating” is also intended to include an increase in the average life expectancy of a diabetic individual compared to a non-treated individual. Other pathological conditions, chronic complications or phenotypic manifestations of the disease are known to those skilled in the art and can similarly be used as a measure of treating diabetes so long as there is a reduction in the severity of the condition, complication or manifestation associated with the disease.

The term preventing refers to a forestalling of a clinical symptom indicative of diabetes. Such forestalling includes, for example, the maintenance of normal levels of blood glucose in an individual at risk of developing diabetes prior to the development of overt symptoms of the disease or prior to diagnosis of the disease. Therefore, the term “preventing” includes the prophylactic treatment of individuals to guard them from the occurrence of diabetes. Preventing diabetes in an individual is also intended to include inhibiting or arresting the development of the disease. Inhibiting or arresting the development of the disease includes, for example, inhibiting or arresting the occurrence of abnormal glucose metabolism such as the failure to transfer glucose from the plasma into the cells. Therefore, effective prevention of diabetes would include maintenance of glucose homeostasis due to glucose-regulated insulin expression in an individual predisposed to a diabetic condition, for example, an obese individual or an individual with a family history of diabetes. Inhibiting or arresting the development of the disease also includes, for example, inhibiting or arresting the progression of one or more pathological conditions or chronic complications associated with diabetes. Examples of such pathological conditions associated with diabetes are listed in Table 1.

The invention provides a mouse comprising a progeny from mating a C57BL/6 mouse and a DBA/2 mouse and exhibiting a blood glucose level of at least about 200 mg/dl, wherein the mouse is a non-human mammalian model predictive of human type 2 diabetes. This mouse model of human type 2 diabetes is referred to herein as a T2D mouse.

Diabetes mellitus is a complex metabolic derangement, which is characterized by either relative or absolute insulin deficiency. The characteristic symptoms of diabetes are polyuria, urinary incontinence, increasing thirst, increasing appetite, tiredness, weight loss and hyperglycaemia. The incidence of diabetes in the overall North American population reaches about 6 percent. Hyperglycaemia is the most characteristic symptoms of diabetes and can be caused by the increase in the rate of glucose production by the liver or decrease in the rate of glucose use by peripheral tissues.

Type II diabetes is also known as non-insulin-dependent diabetes mellitus (NIDDM). Most cases of NIDDM appear during the later decades of life, although the disease is sometimes seen in the maturity onset diabetes of the young (MODY). Familial aggregation and the high concordance rate for the disease (60-100%) in identical twins suggest that genetic factors play an important role in the pathogenesis of NIDDM. A genetic predisposition (e.g., genes involved in insulin resistance, obesity genes, and genes involved in β-cell function) is generally believed to be required for the development of NIDDM. In most genetically predisposed individuals, there is a slow progression from a normal state to insulin resistance, hyperinsulinemia, glucose desensitization, defects in insulin secretion, impaired glucose tolerance, and then to hyperglycaemia. This progressive pathogenesis depends on an interaction between genetic and environmental factors involved in both the initiation and progression of NIDDM.

The type 2 diabetic non-human mammal of the invention exhibits elevated blood glucose levels. A non-human mammal reliably exhibiting human type 2 diabetic conditions has been generated using mouse as an exemplary species. The mouse species has been termed the “T2D” mouse or the “type 2 diabetes” mouse. However, as described further below, the non-human mammalian model for type 2 diabetes can be produced in any mammalian species, except human, where the appropriate parental phenotypes or genotypes are available.

The T2D mouse was developed from genetically crossing C57BL/6 and DBA/2 mice. The C57BL/6 mouse is a well known mouse strain commercially available from Jackson Laboratory, Bar Harbor, Me., and has phenotypic and genotypic characteristics beneficial for producing a non-human mammalian mouse model of human type 2 diabetes. The C57BL/6 mouse is homozygous for Cdh23^{ahl} and has a b H2 haplotype. Further information regarding the characteristics of this mouse strain can be found at the Jackson Laboratory URL: jaxmice.jax.org/jaxmice-cgi/jaxmicedb.cgi?objtype=pricedetail&stock=000664. The DBA/2 mouse also is a well known mouse strain commercially available from Jackson Laboratory, Bar Harbor, Me., that has phenotypic and genotypic characteristics beneficial for producing a non-human mammalian mouse model of human type 2 diabetes. The DBA/2 mouse also is homozygous for Cdh23^{ahl} but has a d H2 haplotype. Further information regarding the characteristics of this mouse strain can be found at the Jackson Laboratory URL: jaxmice.jax.org/jaxmice-cgi/jaxmicedb.cgi?objtype=pricedetail&stock=000671. Mating the C57BL/6 mouse with a DBA/2 mouse produces F₁ progeny that are back crossed with a parental DBA/2 mouse. The resultant progeny are phenotypically screened for elevated levels of blood glucose levels and those exhibiting greater than about 200 mg/dl correspond to progeny where the mating resulted in a genotype conferring type 2 diabetes onto the T2D mouse.

The type 2 diabetic T2D mouse of the invention also can exhibit glucose levels greater than about 200 mg/dl. A level of about 200 mg/dl is indicative of diabetes. As exemplified further below in the Examples, the backcrossed progeny exhibited a significant frequency of glucose level elevated to greater than 250 mg/dl and were used for further characterization of the type 2 diabetic phenotype. However, a type 2 diabetic T2D mouse of the invention can have elevated glucose ranges above about 200 mg/dl and below 250 mg/dl and will still exhibit reliable indicators of human type 2 diabetes. Similarly, a type 2 diabetic T2D mouse of the invention also can be produced that exhibits elevated glucose greater than about 250 mg/dl and which will exhibit reliable indicators of human type 2 diabetes. Therefore, a T2D mouse of the invention can exhibit elevated blood glucose levels of at least about 200, 210, 220, 230, 240, 250, 260, 270, 280, 290, 300 mg/dl or more. Higher levels can include, for example, blood glucose levels of at least about 325, 350, 375, 400 mg/dl or more. T2D mice exhibiting any of these blood glucose level phenotypes will develop diabetes at old age, or at about 25 weeks of age or older.

The type 2 diabetic T2D mouse model of the invention also exhibits a number of additional reliable indicators of type 2 diabetes. Such other indicators include, for example, impaired glucose tolerance, insulin resistance in older mammals, impaired adipocyte or muscle glucose transport and/or decreased expression of at least one polypeptide involved in insulin action. The diminution in polypeptide expression levels can include, for example, insulin receptor (IR)-β subunit, insulin receptor substrate (IRS)-1 and the glucose transporter (GLUT)-4 in adipose tissue. In muscle tissue, diminution in insulin related polypeptide expression can include, for example, the IRS-1. Altered expression of other insulin related polypeptides also can occur due to the diabetic phenotype of the T2D mouse. Those skilled in the art will know or can determine which other insulin related polypeptides can be altered due to the diabetic nature of the mouse and similarly use such other changes in insulin related polypeptide expression as a further characterization of the T2D mouse.

As described further below, any of the above changes in phenotypes compared to a normal mammal can be used to assess the degree of type 2 diabetes in the T2D mouse. Assessment of a single phenotype is sufficient. However, two or more phenotypes also can be assessed to confirm the type 2 diabetic nature of the T2D mouse of the invention, including all combinations of the above characteristics. Similarly, reversion of any of these changed phenotypes in the direction of more normal levels also can be used to assess the efficacy of a therapeutic treatment or to identify test compounds or molecules that have potential therapeutic activity. Insulin production in the T2D mouse can be, for example, normal or increased compared to non-diabetic mice.

Exemplary ranges for the above indicators of type 2 diabetes in the T2D mouse of the invention can vary without the departure from the usefulness of the T2D mouse as a reliable indicator of human type 2 diabetes. For example, impaired glucose tolerance can range from about 200-500 mg/dl after 2 hours of glucose loading, particularly from about 300-500 mg/dl after 2 hours of glucose loading and more particularly from about 400-500 mg/dl after 2 hours of glucose loading. An exemplary level of glucose tolerance impairment is about 300-400 mg/dl after 2 hours of glucose loading.

Similarly, insulin resistance of older mammals also can vary while still being a reliable indicator of human type 2 diabetes in the T2D mouse model. For example, insulin resistance in a T2D mouse of the invention can range from about 70-100% blood glucose decrease after 15 minutes of insulin injection, particularly from about 80-100% blood glucose decrease after 15 minutes of insulin injection and more particularly from about 90-100% blood glucose decrease after 15 minutes of insulin injection. An exemplary level of insulin resistance is about 90-100% blood glucose decrease after 15 minutes of insulin injection.

Further, impaired glucose transport also can vary in the T2D mice of the invention while still maintaining their ability to be a reliable indicator of human type 2 diabetes. For example, impaired glucose transport in adipocytes of a T2D mouse of the invention can range from about 100-200% insulin-stimulated glucose transport, particularly from about 100-180% insulin-stimulated glucose transport and more particularly from about 100-150% insulin-stimulated glucose transport. An exemplary level of impaired adipocyte glucose transport is about 100-150% insulin-stimulated glucose transport. Impaired glucose transport in muscle of a T2D mouse of the invention can range from about 100-150% insulin-stimulated glucose transport, particularly from about 100-140% insulin-stimulated glucose transport and more particularly from about 100-130% insulin-stimulated glucose transport. An exemplary level of impaired glucose transport in muscle is about 110-130% insulin-stimulated glucose transport.

Decreased expression of IR-β, IRS-1 and GLUT-4 also can vary without the T2D mouse of invention departing from a reliable human type 2 diabetic mammalian model. For example, the diminution in expression levels can range from about 2-10-fold, particularly from about 3-7-fold and more particularly from about 4-6-fold. An exemplary decrease in expression if about 5-fold. Similarly, for insulin related polypeptides that are low in the normal state and increase in a diabetic state, the increased level of this class of polypeptide also can range from about 2-10-fold, particularly from about 3-7-fold and more particularly from about 4-6-fold. An exemplary decrease in expression if about 5-fold.

The invention also provides a method of producing a non-human mammal indicative of type 2 diabetes. The method includes mating a C57BL/6 mouse with a DBA/2 mouse, and backing crossing offspring of the mating with a parental DBA/2 mouse to produce a progeny exhibiting a blood glucose level of at least about 200 mg/dl that is predictive of human type 2 diabetes.

As described previously, the T2D mouse was developed from genetically crossing C57BL/6 and DBA/2 mice. The progeny of this mating was then backcrossed to a parental DBA/2 mouse to produce the T2D mouse having a type 2 diabetes phenotype This same genetic methodology also can be used to produce non-human mammals from species other than murine. For example, a parental non-human mammal having phenotypic characteristics and/or a genotype substantially corresponding to the C57BL/6 mouse, can be mated with a non-human mammal having phenotypic characteristics and/or a genotype substantially corresponding to the DBA/2 mouse, to produce a first progeny. Mating the first progeny of this cross with the non-human mammal having phenotypic and/or a genotype substantially corresponding to the DBA/2 mouse will generate a litter of non-human mammals indicative of human type 2 diabetes, and similar to the T2D mouse litter described herein. The type 2 diabetic non-human mammals can be confirmed to have a diabetic phenotype by confirming a blood glucose level of at least about 200 mg/dl and/or confirming one or more of the other related phenotypic characteristics described previously. As with the T2D mouse, a non-human type 2 diabetic model of the invention can be used to predict the onset, occurrence or progression, for example, of human type 2 diabetes.

Species applicable for producing a non-human mammalian model of human type 2 diabetes include, for example, rat and monkey as well as other mammalian species well known in the art. As with the mouse model described previously, a human type 2 diabetes model produced from non-mouse species also will exhibit one or more of the diabetic indicators including, for example, impaired glucose tolerance, insulin resistance in older mammals, impaired adipocyte or muscle glucose transport and/or decreased expression of at least one polypeptide involved in insulin action.

The non-human mammals indicative of type 2 diabetes are useful in the design and efficacy assessment of therapeutic treatments. The therapeutic treatments can include insulin therapies and combinations of insulin therapies to assess, for example, whether a change in the therapeutic regime is warranted or enhanced compared to control conditions. Additionally, the non-human type 2 diabetes models of the invention also can be employed as recipients of gene therapy or cell therapy protocols to determine whether a particular gene, a combination of genes, unmodified cells and/or genetically modified cells reduce symptoms of diabetes or augments insulin therapy. The non-human type 2 diabetic mammals of the invention can be genetically modified as desired or cells can be implanted having desired characteristics and the modified non-human mammal of the invention can be assessed for a reduction in the severity of one or more diabetic symptoms. Diabetic symptoms applicable an as indicators include those described previously as well as those listed in Table 1, for example. The non-human mammals indicative of type 2 diabetes also are useful to identify therapeutic compounds effective for treating, preventing or curing type 2 diabetes. Methods for identifying therapeutic compounds are exemplified further below.

The invention further provides method of screening for a therapeutic agent for use in treating type 2 diabetes. The method includes: (a) administering a compound to a T2D mouse of the invention, and (b) screening the mouse for a reduced symptom of type 2 diabetes, thereby identifying a therapeutic agent for use in treating type 2 diabetes. The therapeutic agents of the invention can be macromolecules such as polypeptides or nucleic acids and/or organic compounds such as receptor ligands, agonists and/or antagonists. The therapeutic agents identified by the method of screening of the invention can be used in the treatment of human type 2 diabetes.

The non-human mammal indicative of human type 2 diabetes of the invention can be advantageously used to screen for therapeutic agents that can be used to treat type 2 diabetes. For example, a non-human type 2 diabetic mammal of the invention, including a T2D mouse, exhibits elevated blood glucose levels of at least about 200 mg/dl. The invention thus provides a method of identifying a therapeutic agent for use in treating human type 2 diabetes by administering a compound to a non-human mammal having phenotypic characteristics and/or a genotype substantially corresponding to a progeny of a genetic cross between C57BL/6 and DBA/2 mice and screening the non-human mammal for reduced symptoms of diabetes. A reduction in one or more symptoms of diabetes identifies a therapeutic agent for use in treating this disease. One skilled in the art can readily determine whether a reduction in type 2 diabetic symptoms occurs given the teachings and guidance provided herein.

As described in Example I, in addition to exhibiting elevated blood glucose levels, the T2D mouse and other non-human mammals of the invention also exhibit impairment of glucose tolerance and transport, insulin resistance and altered expression of several genes in comparison to animals. For example, the T2D mice are characterized by decreased expression of IR-β, IRS-1 and/or GLUT-4. The altered expression of each of these genes, as well as other genes having altered expression in a non-human type 2 diabetic mammal of the invention, indicates that these genes can be used as a readout indicator to assess efficacy of a test compound for treating human type 2 diabetes. Blood glucose levels, glucose tolerance, glucose transport and/or insulin resistance also can be used as a readout indicator to assess efficacy of a test compound for treating human type 2 diabetes.

The methods of the invention for screening for a therapeutic agent for use in treating type 2 diabetes involve administering a test compound to a non-human type 2 diabetic mammal such as the T2D mouse. A test compound can be any substance, molecule, compound, mixture of molecules or compounds, or any other composition which is suspected of being capable of restoring one or more phenotypes or symptoms of diabetes described herein or known in the art to a more normal level.

A test compound can be a macromolecule, such as biological polymer, including polypeptides, polysaccharides and nucleic acids. Compounds useful as potential therapeutic agents can be generated by methods well known to those skilled in the art, for example, well known methods for producing pluralities of compounds, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

Additionally, a test compound can be preselected based on a variety of criteria. For example, suitable test compounds having known modulating activity on a pathway known or suspected to be involved in diabetes, and particularly in type 2 diabetes, can be selected for testing in the screening methods. Alternatively, the test compounds can be selected randomly and tested by the screening methods of the present invention. Test compounds can be administered to the non-human type 2 diabetic mammal of the invention at a single concentration or, alternatively, at a range of concentrations from about 1 nM to 1 mM or higher.

The number of different test compounds examined using the methods of the invention will depend on the application of the method. It is generally understood that the larger the number of candidate compounds, the greater the likelihood of identifying a compound having the desired activity in a screening assay. The methods can be performed in a single or multiple sample format. Large numbers of compounds can be processed in a high throughput format which can be automated or semi automated.

Following administration of a test compound, one or more indicators of diabetes is measured. Those test compounds that reduce one or more phenotypes or symptoms of diabetes as described herein is identified as a therapeutic agent for use in treating type 2 diabetes. Reduction in a phenotype or symptom of diabetes includes, for example, decreased blood glucose levels, increased glucose tolerance, increase glucose transport and/or decreased insulin resistance. Additionally, reduction in one or more of the symptoms set forth in Table 1 also will identify the test compound as a therapeutic agent for use in treating type 2 diabetes. The restoration of expression levels for genes or polypeptides involved in insulin action also can be measured as an indicator of a therapeutic agent useful for treating type 2 diabetes.

Measuring the gene or polypeptide expression levels of target genes such as those involved in insulin action can be particularly useful for high throughput screening of potential therapeutic compounds. For example, the expression of a gene involved in insulin action, or the modulation of expression of a target gene by a test compound, can be determined by measuring changes in expression. This method of screening of the invention involves measuring changes in gene expression by determining the amount of mRNA or polypeptide present in a sample. Methods for measuring both MRNA and polypeptide quantity are well known in the art. Methods for measuring MRNA typically involve detecting nucleic acid molecules by specific hybridization with a complementary probe in solution or solid phase formats. Such methods include northern blots, polymerase chain reaction after reverse transcription of RNA (RT PCR), and nuclease protection. Measurement of a response of a pathway component can be performed using large scale gene expression methods.

Large scale gene expression methods can be advantageously used to measure a large population of expressed genes in an organ, tissue or cell. Examples of methods well known in the art applicable to measuring a change in expression of a population of genes include cDNA sequencing, clone hybridization, differential display, subtractive hybridization, cDNA fragment fingerprinting, serial analysis of gene expression (SAGE), and DNA microarrays. These methods are useful, for example, for identifying differences in gene expression in an organ, tissue or cell of a non-human type 2 diabetic mammal of the invention compared to that of a control mammal. Methods of detecting changes in gene expression can be performed both qualitatively or quantitatively.

A level of protein expression corresponding to a gene expression level also can be determined, if desired. A variety of methods well known in the art can be used to determine protein levels either directly or indirectly. Such methods include immunochemical methods, such as western blotting, ELISA, immunoprecipitation, and RIA, gel electrophoresis methods including one and two-dimensional gels, methods based on protein or peptide chromatographic separation, methods that use protein fusion reporter constructs and colorimetric readouts, methods based on characterization of actively translated polysomal MRNA, and mass spectrometric detection.

The methods of the invention for identifying a therapeutic agent also can include determining an activity of a target gene. The activity of a molecule can be determined using a variety of assays appropriate for the particular target. A detectable function of a target gene such as one or more genes involved in insulin action can be determined based on known or inferred characteristics of the target gene. For example, receptor binding function can be assessed by measuring a known physiological response following ligand binding.

Compounds identified as therapeutic agents by methods of the invention can be administered to an individual, for example, to alleviate a sign or symptom associated with diabetes, particularly type 2 diabetes, or any phenotype associated with a non-human type 2 diabetic mammal of the invention. One skilled in the art will know or can readily determine the alleviation of a sign or symptom associated with type 2 diabetes given the teachings and guidance provided herein.

If desired, appropriate control animals can be used to corroborate the therapeutic effectiveness of screened compounds. For example, a control animal can be one that exhibit normal levels for blood glucose, glucose tolerance, insulin resistance, glucose transport and/or expression levels of polypeptides involved in insulin action. Such normal levels are known to those skilled in the art and exemplified, for example, by the control animals in described below in Example I.

For use as a therapeutic agent, the compound can be formulated with a pharmaceutically acceptable carrier to produce a pharmaceutical composition, which can be administered to the individual, which can be a human or other mammal. A pharmaceutically acceptable carrier can be, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester. A pharmaceutically acceptable carrier can also contain physiologically acceptable compounds that act, for example, to stabilize or increase the absorption of the modulatory compound. One skilled in the art would know that the choice of a pharmaceutically acceptable carrier, including a physiologically acceptable compound, depends, for example, on the route of administration of the composition.

The methods of the invention can advantageously use cells isolated from a non-human type 2 diabetic mammal of the invention, including the exemplified T2D mouse of the invention. Such cells can be used as an in vitro method to screen compounds as potential therapeutic agents for treating human diabetes, and particularly human type 2 diabetes. In such a method, a compound is contacted with a cell isolated from a non-human type 2 diabetic mammal of the invention and screened for alterations in a cellular phenotype associated with type 2 diabetes.

Additionally, genes involved in insulin action also can be modulated in order to reverse, or at least partially reverse, the physiological and/or biochemical characteristics of human type 2 diabetes. The non-human type 2 diabetic mammal of the invention can be used to assess which genes or genes are appropriate therapeutic targets that can confer restoration of a more normal phenotype upon therapeutic compound intervention. For example, restoring a more normal level of expression of a target gene having altered expression in a non-human type 2 diabetic mammal of the invention can result in reduced blood glucose levels, increased glucose tolerance, increased glucose transport and/or decreased insulin resistance. Therefore, a compound that restores a more normal level of expression to a target gene having altered expression in a non-human type 2 diabetic mammal of the invention is a potentially useful therapeutic compound for treatment of type 2 diabetes and reduction in the severity of its symptoms. The method for identifying a compound that restores a target gene involved in insulin action includes, selecting a target gene having altered expression in a non-human type 2 diabetic mammal; contacting a target gene having altered expression in a non-human type 2 diabetic mammal with a test compound; (b) determining expression of said target gene, and (c) identifying a compound that modulates expression of said target gene to a level of expression consistent with a normal level of expression.

A more normal level of expression of a target gene is a level of expression of the target gene that is similar to the level of expression of the target gene in a non-diabetic animal. A test compound that restores a target gene having altered expression in a non-human type 2 diabetic mammal of the invention to a more normal level of expression changes the level of expression to at level at least about 50% toward the normal level of expression in a non-human non-diabetic mammal, intact cell or cell preparation. For example, a test compound that restores a target gene have reduced expression in a non-human type 2 diabetic mammal to a more normal level of expression does so by increasing the level of expression by at least about 50% toward the normal level of expression. Likewise, a test compound that restores a target gene having increased expression in a non-human type 2 diabetic mammal to a more normal level of expression does so by decreasing the level of expression by at least about 50% toward the normal level of expression. For example, a test compound can restore at least about 60%, at least about 70%, at least about 80%, at least about 90%, at least about 95%, at least about 98%, or even essentially about 100% of the normal level of expression in a non-human non-diabetic mammal.

A target gene having altered expression can be contained, for example, in a non-human type 2 diabetic mammal of the invention, in an organ, tissue or cell isolated therefrom, or in a cell preparation in which expression of a target gene can be modulated.

It is understood that modifications which do not substantially affect the activity of the various embodiments of this invention are also included within the definition of the invention provided herein. Accordingly, the following examples are intended to illustrate but not limit the present invention.

### EXAMPLE I

### Non-Insulin Dependent Diabetes Non-Human Mouse Model

This Example shows the generation and characterization of a non-insulin dependent non-human mouse model of Type 2 diabetes.

A mouse model for human type 2 diabetes, termed the T2D mouse, was produced by mating the C57BL/6 mouse and DBA/2 mouse. The C57BL/6 mouse is a well known inbred strain commercially available from Jackson Laboratory, Bar Harbor, Me. This mouse is homozygous for Cdh23^{ahl} and has a b H2 haplotype. Further information regarding the characteristics of this mouse strain can be found at the Jackson Laboratory URL: jaxmice.jax.org/jaxmice-cgi/jaxmicedb.cgi?objtype=pricedetail&stock=000664. Similarly, the DBA2 mouse also is a well known inbred strain commercially available from Jackson Laboratory, Bar Harbor, Me. This mouse also is homozygous for Cdh23^{ahl} but has a d H2 haplotype. Further information regarding the characteristics of this mouse strain can be found at the Jackson Laboratory URL: jaxmicejax.org/jaxmice-cgi/jaxmicedb.cgi?objtype=pricedetail&stock=000671. Mating was performed using well known method in the art. Progeny of the C57BL/6×DBA/2 cross were further backcrossed by mating with DBA/2 parental mice.

The incidence of diabetes in T2D mice at 30 weeks of age was determined by measuring blood glucose level. Blood glucose was measured via the glucose oxidase method, using tail-blood and a One-Touch Profile portable blood glucose monitor (Lifescan, Milpitas Calif.).

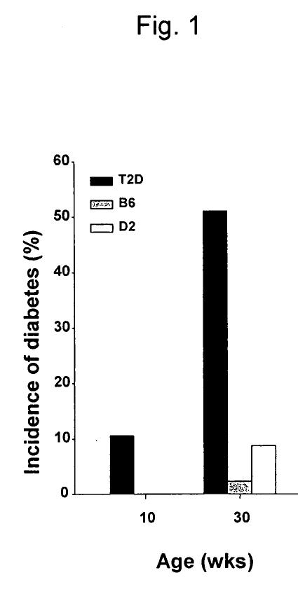

The animals with blood glucose level more than 250 mg/dl were considered as diabetes. FIG. 1 shows the incidence of diabetes observed in the T2D mouse. Mice exhibiting greater than 250 mg/dl were found to occur in 51% (24/47) percentage of the backcrossed T2D mice. Less than 10% of control mice developed diabetes (C57BL/9: 2.2%, DBA2: 8.7%). Even at 10 wks of ages, T2D mice showed 10.6% of diabetes incidence when none of the control mice became diabetes.

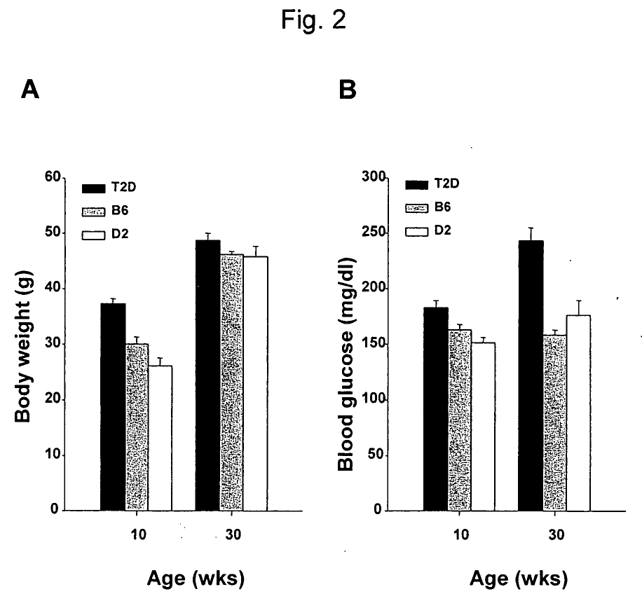

FIG. 2 represents incidence of diabetes with respect to body weight and blood glucose levels in the T2D mice. The results show that the body weight of T2D mice at 10 wks of age was higher than control groups but at 30 wks of age body weight of T2D mice was not significantly different from the control mice (FIG. 2A). The blood glucose level of T2D mice was slightly higher at 10 wks of age compared to control mice. Moreover, at 30 wks of age, blood glucose levels in T2D mice were significantly higher than that of control groups (FIG. 2B).

The offspring of two mice showed hyperglycemia, having a blood glucose level of more than 250 mg/dl. These offspring also were insulin resistance and exhibited impaired glucose tolerance at old age, which was at measured at 30 weeks of age. Insulin secretion was higher in T2D mice compared to the above control mice. The phenotype of hyperglycemia observed in the offspring is similar to type 2 diabetes.

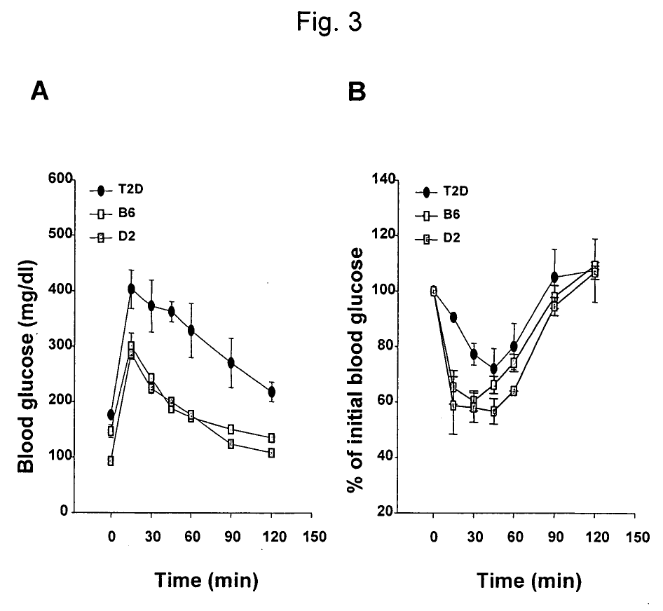

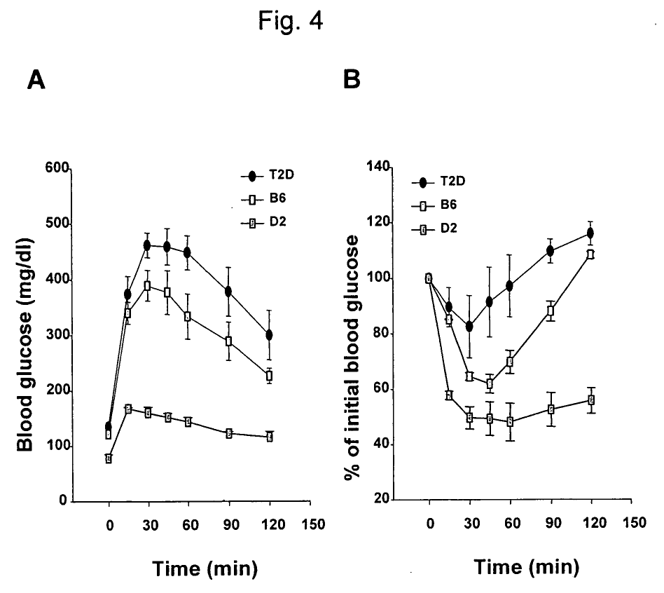

Characterization of the T2D mouse with respect to glucose tolerance and insulin tolerance also was examined at 10 and 30 weeks of age. Briefly, glucose tolerance was examined by fasting mice overnight (>15 hrs) and administering glucose by intraperitoneal (i.p.) injection with glucose at a dose of 2 g/kg per body weight). The blood glucose levels were measured as described above at 0, 15, 30, 60, 90 and 120 minutes after glucose injection (Lee et al., Nature 408:483-488 (2000). FIG. 3 shows glucose tolerance levels in T2D mice at 10 weeks of age. FIG. 4 shows glucose tolerance at 30 weeks of age.

The results indicate that at 10 wks of age, T2D mice can not efficiently clear the glucose in the blood indicating that the glucose tolerance is impaired in T2D mice compared to the control groups (FIG. 3A). At 30 wks of age, T2D mice showed severe impairment of glucose tolerance while C57BL/6 mice also shows impaired glucose tolerance (FIG. 4A). DBA2 mice however, showed normal clearance of glucose from the blood. Although C57BL/6 mice showed impaired glucose tolerance, they did not develop overt diabetes measured by blood glucose level.

To examine the insulin tolerance, mice were injected i.p. with human insulin at 1 unit/kg of body weight. The blood glucose levels were measured at 0, 15, 30, 60, 90 and 120 minutes after insulin injection for both 10 week and 30 week old mice. The insulin tolerance results are shown in FIGS. 3 and 4, respectively. At 10 wks of age, the blood glucose level of T2D mice was not efficiently decreased after insulin injection as in the control groups (FIG. 3B). This result indicates that T2D mice have weak insulin resistance resulting in the slightly hyperglycemic condition. At 30 wks of age, T2D mice showed almost no responsiveness to insulin indicating a severe insulin resistance state (FIG. 4B). C57BL/6 mice also showed slight insulin resistance but less than that of T2D. Because insulin action in C57BL/6 mice is almost normal, the blood glucose level of C57BL/6 is almost normal in physiological condition although glucose can not be cleared efficiently after artificial load.

Insulin production in the T2D mice also was assessed by immunohistochemistry. Briefly, pancreas was removed from 30 wks old control and T2D mice and stained with anti-insulin antibody. The samples were fixed with 10% buffered formalin, embedded in paraffin, sectioned at 4.5 μm, and mounted on glass slides. The samples were treated with xylene, and 100%, 90%, 80%, and 70% ethanol in sequence and washed with tap water. Non-specific antibody binding was blocked using blocking buffer (1% (w/v) BSA, 0.2% (v/v) Tween-20 in PBS) for 1 hour. Primary antibody (guinea pig anti-rat insulin (DAKO, Carpiteria, Calif., USA)) was diluted in blocking buffer (1/200) and applied to cells for 1 hour. Samples were washed 3 times in PBS. Secondary antibody (biotinylated anti-guinea pig antibody) was diluted in blocking buffer (1/300) and added to cells 1 hour. After 3 times wash, HRP-conjugated streptavidin was diluted in blocking buffer (1/300) and added to cells 1 more hour, followed by colour developing using Vector VIP (Vector laboratory, Burlingame, Calif., USA) according to manufacturer's instruction. After washing with tap water samples were counterstained with Meyer hematoxyln solution.

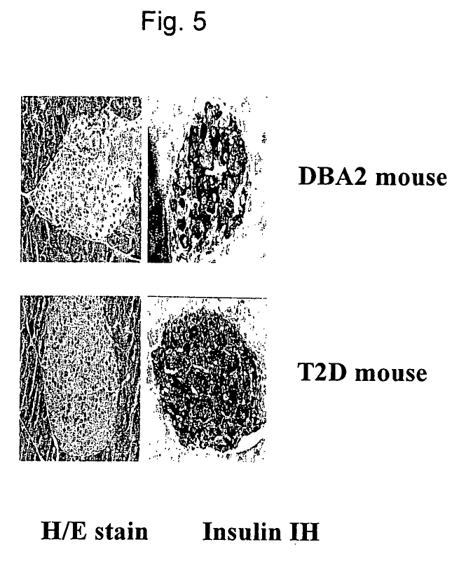

FIG. 5 shows insulin immunohistostaining results of pancreatic islet cells and reveals no obvious lesion in pancreas examined by H/E staining. By immunohistochemical staining of insulin, it was found that pancreas from T2D mouse has equal or higher amount of insulin indicating that insulin production was not impaired in pancreatic β-cells of T2D mice.

Insulin secretion was measured by radioimmunoassay of C-peptide which secreted along with insulin by pancreatic β-cells. Briefly, blood samples were collected from fasted mice, non-fasted mice and the mice were tested glucose tolerance as described previously. Immunoreactive C-peptide was measured with radioimmunoassay kit for mouse C-peptide (Linco Research, Inc, St. Charles, Mich.).

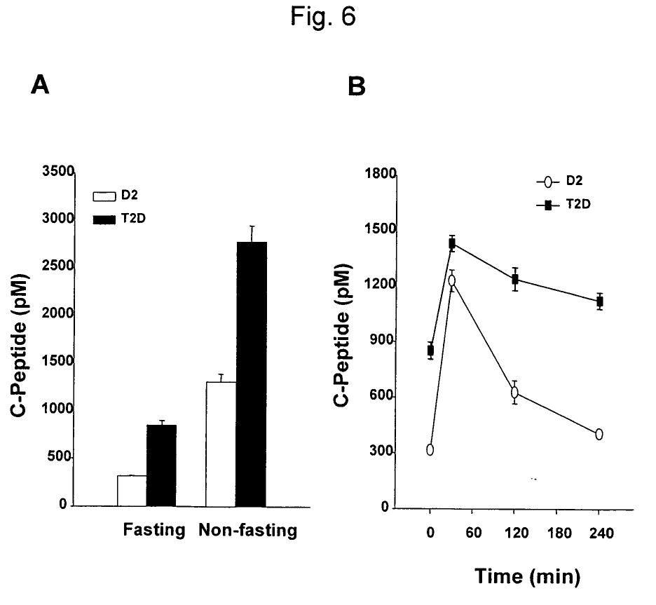

The results of this analysis are shown in FIG. 6 and indicate that C-peptide level in fasted or non-fasted T2D mice was higher than that of control mice (FIG. 6A). Furthermore, the secretion of C-peptide after glucose loading was also higher in T2D mice than in control mice (FIG. 6B). Taken the above results together, in T2D mice, insulin production and secretion is normal or higher than that of control mice.

Glucose transport in adipose tissue and muscle were measured in T2D mice at 10 and 30 wks of age. Adipocytes were isolated and incubated with insulin. Muscle (soleus muscle) was isolated from the legs and incubated with insulin. Glucose transport was measured by radioactive glucose analogue 2-deoxyglucose.

The ability of adipocytes from T2D mice to transport glucose also was characterized. Briefly, epididymal adipose tissues were removed from 10 and 30 week old T2D mice and digested with collagenase type II (1 mg/ml; Sigma) in 1 ml Krebs-Ringer HEPES (KRH) buffer (131 mM NaCl, 4.7 mM KCl, 2.5 mM CaCl2, 1.25 mM MgCl2, 2.5 mM NaH2PO4, 10 mM HEPES, pH 7.4) supplemented with 1% BSA and 2 mM pyruvate for 1 hour at 37° C. in shaking water bath as described by Olefsky J. M., J Clin. Invest. 56:1499-1508 (1975). After digestion, adipocytes were liberated by gentle massage with rubber policeman followed by sieving through 350 μm polypropylene mesh. Isolated adipocytes were washed with fresh KRH buffer without collagenase three times. The number of adipocytes was counted under the microscope by the method of Green (Green A., Biochem. J. 238:663-669 (1986)).

Adipocytes (3-4×105/ml) suspended in KRH buffer were placed in plastic vial and incubated for 20 min at 37° C. in shaking water bath. After incubation, adipocytes were treated with 25 ng/ml insulin for 30 min. After incubation, [3^{H}]-2-deoxyglucose (2-DOG; 0.5 μCi, 0.125 mM; Amersham Pharmacia Biotech, Buckinghamshire, UK) was added and cells were incubated for 3 minutes. 300 μl of cell suspension was taken from the reaction medium and put into microtubes containing 100 μl of silicone oil (Sigma). Cells were centrifuged for 30 seconds at full speed and the oil layer was cut after centrifugation (Khil et al., Biochem. Pharmacol. 58:1705-1712 (1999)). The radioactivity of the cell layer was measured with a Beta-counter. Trapped [3^{H}]-2-DOG on the outer surface of adipocytes was measured in the presence of 1.2 mM phloretin (Sigma) and this radioactivity was subtracted from sample data (Rodriguez et al., Endocrinology 145:679-685 (2004)).

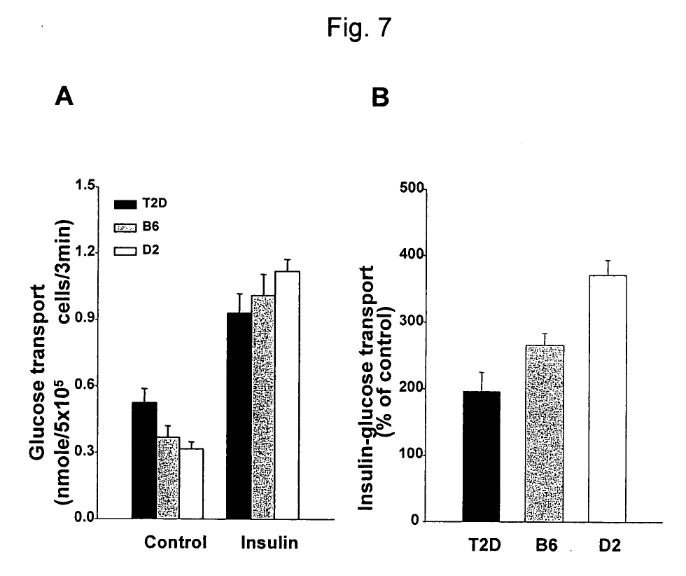

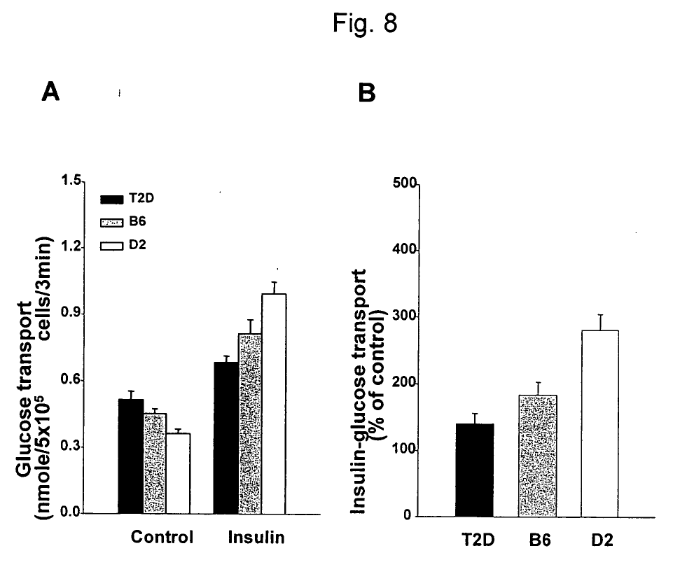

FIG. 7A and FIG. 8A show the real value of glucose transport in adipose tissue in the presence or absence of insulin at 10 or 30 wks of age. Insulin-stimulated glucose transport was calculated by dividing glucose transport in the presence of insulin by glucose transport without insulin and the data are shown in FIG. 7B (at 10 week old mice), while the data from 30 week old mice are shown in FIG. 8B. The results indicate that glucose transport was significantly lower than those of control mice.

In addition to adipocyte glucose transport, the glucose transport ability of muscle tissue from both 10 and 30 week old T2D mice also were assessed. Briefly, muscle strips (soleus muscle) were removed from hind limb and put in KRH after weighing. Muscle strip was gas for 3 min with 95% O2/5% CO2 and incubated for 30 min at 37° C. After incubation muscle strip was transferred to another vial containing KRH and [3^{H}]-DOG in the presence or absence of insulin. After gas pursing, muscle strip was incubated further 30 min. after incubation, muscle strip was washed with ice cold PBS for three times and dissolved in NaOH/KOH for 1 hr. The radioactivity in dissolved muscle was measured by Beta-counter (Rodriguez et al., Endocrinology 145:679-85 (2004)).

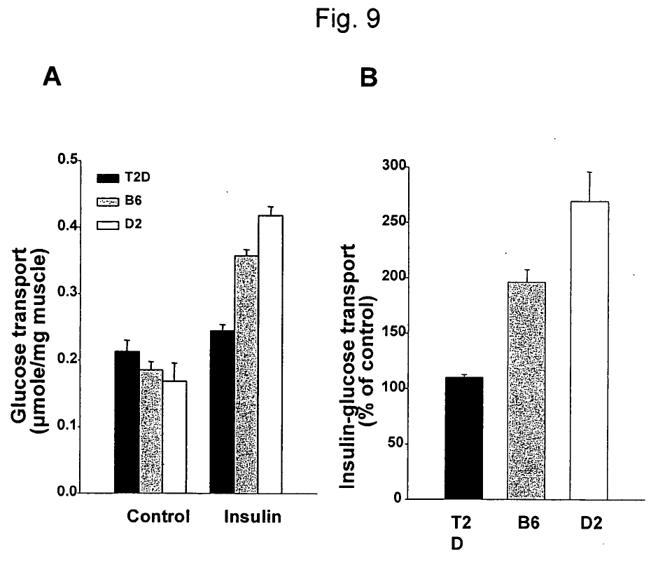

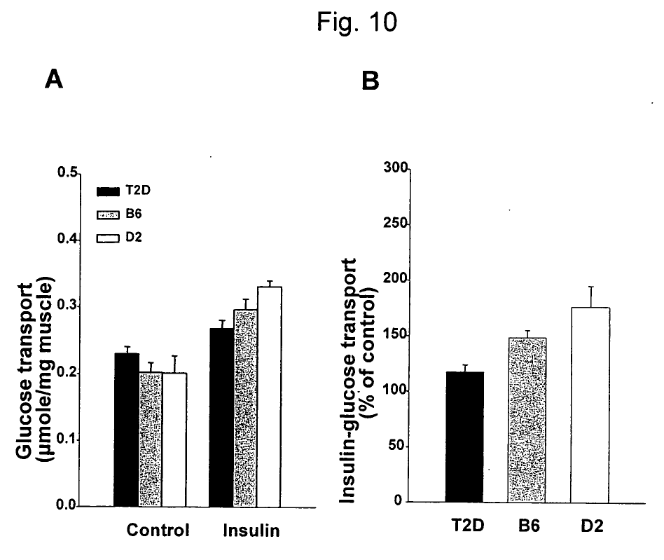

FIG. 9A and FIG. 10A show the real value of glucose transport in muscle in the presence or absence of insulin at 10 or 30 wks of age. Insulin-stimulated glucose transport was calculated by dividing glucose transport in the presence of insulin by glucose transport without insulin and the data are shown in FIG. 9B (at 10 week old mice), while the data from 30 week old mice are shown in FIG. 10B. The results indicate that glucose transport in muscle also was less than that for the control groups. Because glucose in blood is cleared by uptake into cells through glucose transport, impaired glucose transport results in the higher blood glucose level.

The expression of proteins which are required for insulin action on glucose metabolisms were examined in T2D mice. Expression was assessed in both adipose and muscle tissue by western blot.

Briefly, muscle and adipose tissue were removed at certain ages of mice and homogenized with teflon homogenizer in homogenization buffer. Protein contents were determined and protein preparations were boiled in sample buffer for 5 minutes. Proteins were separated by SDS-PAGE with 10% separating gel under 15 mA/gel constant current. After electrophoresis, proteins were transferred to PVDF membrane (Amersham Pharmacia Biotech) with Semi-Dry transblot kit (Bio-Rad, Hercules, Calif.) under 15V constant voltage for 70 minutes. After transfer, the membranes were blocked with 5% skim milk in Tris-buffered saline with tween 20 (TBST; 0.05% tween 20, 200 mM Tris-HCl, 500 mM NaCl, pH 7.5) for 1 hour at room temperature. Western blot protocols depend on the specific antibody used for study. Generally, after blocking of membrane, primary antibodies in 5% BSA in TBST were incubated with the membrane for 1 hour at room temperature or overnight at 4° C. Membrane was washed with TBST for 30 minutes at room temperature. Secondary antibodies labeled with horse reddish peroxidase (HRP) in 5% skim milk in TBST were added and incubated for 1 hour. Membrane was washed with TBST for 30 minutes at room temperature. Immunoreactive proteins were detected with enhanced chemiluminescence western blot detection kit (Amersham Pharmacia Biotech).

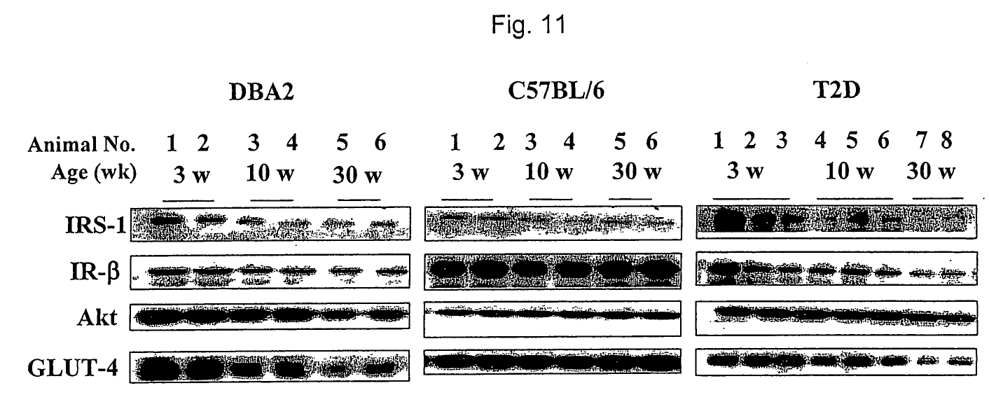

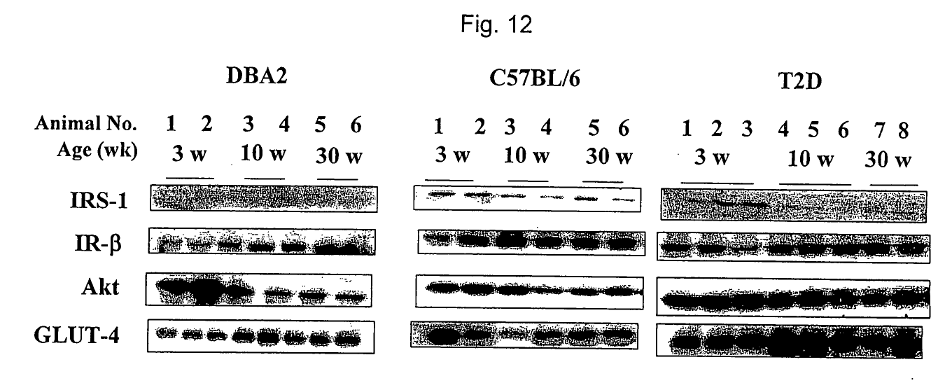

FIG. 11 shows age-dependent expression of proteins required for insulin action in adipose tissue from T2D mice. FIG. 12 shows age-dependent expression of proteins required for insulin action in soleus muscle from T2D mice. In adipose tissue, the results indicate that the insulin receptor (IR)-β, subunit was decreased in T2D mice by age. Insulin receptor substrate (IRS)-1 also was decreased in older T2D mice compared to younger mice. Similarly, glucose transporter (GLUT)-4 also was greatly decreased in older T2D mice. In muscle, IRS-1 was decreased in older T2D mice compared to young animals.

The combined results above indicate that in T2D mice, insulin signaling molecules such as IR-β and IRS-1 were decreased at old age resulting in insulin resistance. Decreased GLUT-4 amount in T2D mice plays a major role in impaired glucose transport and glucose tolerance.

The above results indicate that the T2D mouse can be characterized as not obese compared to C57BL/6 mouse and/or the ob/ob or db/dbmice. The T2D mouse shows moderate to severe hyperglycemia at old age, which corresponds to about 30 weeks of age. Insulin production and secretion were not impaired in T2D mouse. However, insulin-stimulated glucose transport is impaired in adipose tissue and muscle of T2D mouse. Proteins which are very important for insulin stimulated glucose metabolisms are less expressed in T2D mouse. These molecular and cellular disorders contribute to develop insulin resistance and overt diabetes in T2D mice. Accordingly, the T2D mouse has symptoms of type 2 diabetes similar to human diabetes and can be used as a reliable model for the study of diabetes and development of anti-diabetic agents.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A mouse comprising a progeny from mating a C57BL/6 mouse and a DBA/2 mouse and exhibiting a blood glucose level of at least about 200 mg/dl, wherein said mouse is a non-human mammalian model predictive of human type 2 diabetes.

2. The mouse of claim 1, further comprising impairment of glucose tolerance.

3. The mouse of claim 1, further comprising insulin resistance at about 30 weeks of age.

4. The mouse of claim 1, further comprising normal or increased insulin production.

5. The mouse of claim 1, further comprising impaired adipocyte or muscle glucose transport.

6. The mouse of claim 1, further comprising decreased expression of at least one polypeptide involved in insulin action.

7. A method of producing a non-human mammal predictive of type 2 diabetes, comprising mating a C57BL/6 mouse with a DBA/2 mouse, and backing crossing offspring of said mating with a parental DBA/2 mouse to produce a progeny exhibiting a blood glucose level of at least about 200 mg/dl that is predictive of human type 2 diabetes.

8. The method of claim 7, wherein said non-human mammal further comprises impairment of glucose tolerance.

9. The method of claim 7, wherein said non-human mammal further comprises insulin resistance at about 30 weeks of age.

10. The method of claim 7, wherein said non-human mammal further comprises normal or increased insulin production.

11. The method of claim 7, wherein said non-human mammal further comprises impaired adipocyte or muscle glucose transport.

12. The method of claim 7, wherein said non-human mammal further comprises decreased expression of at least one polypeptide involved in insulin action.

13. The method of claim 7, wherein said non-human mammal comprises a mouse or a rat.

14. A method of screening for a therapeutic agent for use in treating type 2 diabetes, comprising:
(a) administering a compound to the mouse of claim 1, and
(b) screening said mouse for a reduced symptom of type 2 diabetes, thereby identifying a therapeutic agent for use in treating type 2 diabetes.

15. The method of claim 14, wherein said reduced symptom comprises increased glucose tolerance.

16. The method of claim 14, wherein said reduced symptom comprises decreased insulin resistance.

17. The method of claim 14, wherein said reduced symptom comprises increased adipocyte or muscle glucose transport.

18. The method of claim 14, wherein said reduced symptom comprises increased expression of at least one polypeptide involved in insulin action.
